(19) United States
(12) Patent Application Publication
(10) Pub. No.: US 2005/0129768 A1
(43) Pub. Date: Jun. 16, 2005

(54) Pharmaceutical formulation containing an LTB4-antagonist, as well as processes for the preparation thereof and the use thereof

(75) Inventors: Thomas Bock, Copenhagen S. (DK); Siglinde Moll, Schemmerhofen (DE); Karl Weber, Grodt-Ingoldingen (DE); Ulrich Brauns, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 10/977,035

(22) Filed: Oct. 29, 2004

(30) Foreign Application Priority Data
Oct. 29, 2003 (DE) 103 50 528

(51) Int. Cl. A61K 31/325; A61K 9/14

(52) U.S. Cl. 424/486; 514/476

(57) ABSTRACT

The invention relates to a new pharmaceutical formulation, containing an LTB₄ antagonist of formula I 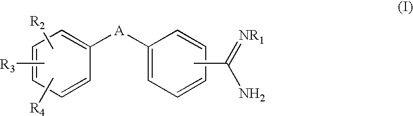
wherein A, R₁, R₂, R₃ and R₄ are defined as in claim 1, the pharmacologically acceptable acid addition salt, glycoside, O-sulphate or glucuronide thereof as active substance as well as optionally at least one pharmacologically acceptable excipient and/or carrier, the active substance being present as a solid solution or solid dispersion in a polymer matrix. The invention also relates to the preparation thereof and their use as pharmaceutical compositions as well as the solid solutions and dispersions per se.

The invention relates to a new pharmaceutical formulation containing an LTB₄-antagonist which comprises a benzamidine group, processes for the preparation thereof and the use thereof as a medicament.

### BACKGROUND OF THE INVENTION

LTB₄-antagonists which comprise a benzamidine group are compounds with pharmacologically valuable properties. LTB₄-antagonists may have considerable therapeutic benefit for example in the treatment of arthritis, asthma, chronic obstructive pulmonary disease, psoriasis, ulcerative colitis, Alzheimer's disease, shock, reperfusion damage/ischaemia, cystic fibrosis, arteriosclerosis and multiple sclerosis.

Compounds of this kind are known e.g. from International Patent Applications WO 93/16036, WO 94/11341, WO 96/02497, WO 97/21670, WO 98/11062, WO 98/11119, WO 01/25186, PCT/EP01/00262 and WO 03/07922.

These compounds have the chemical structure of formula I: 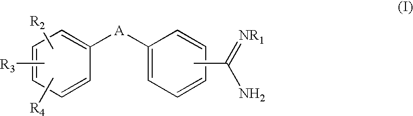
wherein
A denotes a group of formula
   —O—CₘH₂ₘ—O-(PHE)ₙ- (II)
wherein
   m is an integer from 2 to 6, preferably 2 to 5,
   n is 0 or 1,
   PHE denotes a 1,4-phenylene group optionally substituted by one or two C₁-C₆-alkyl groups, preferably a 1,4-phenylene group substituted by a C₂-C₄-alkyl group linked to the oxygen in the ortho-position;
or
A denotes a group of formula 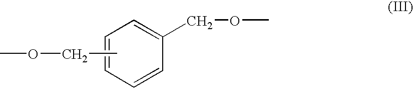
   preferably of formula 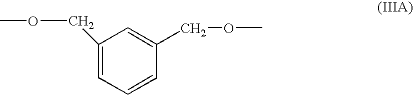
      wherein
R₁ denotes H, OH, CN, COR₁₀, COOR₁₀, or CHO, preferably H or COOR₁₀;
R₂ denotes H, Br, Cl, F, CF₃, CHF₂, OH, HSO₃—O, C₁-C₆-alkyl, C₁-C₆-alkoxy, C₅-C₇-cycloalkyl, CONR₈R₉, aryl, O-aryl, CH₂-aryl, CR₅R₆-aryl, or C(CH₃)₂—R₇, preferably OH, HSO₃—O, CONR₈R₉, or CR₅R₆-aryl,
R₃ denotes H, C₁-C₆-alkyl, C₁-C₆-alkoxy, OH, Cl, or F, preferably H or C₁-C₃-alkoxy,
R₄ denotes H or C₁-C₆-alkyl, preferably H;
R₅ denotes C₁-C₄-alkyl, CF₃, CH₂OH, COOH, or COO(C₁-C₄-alkyl), preferably C₁-C₄-alkyl, particularly methyl;
R₆ denotes H, C₁-C₄-alkyl, or CF₃, preferably C₁-C₄-alkyl, particularly methyl;
R₇ denotes CH₂OH, COOH, COO(C₁-C₄-alkyl), CONR₈R₉, or CH₂NR₈R₉;
R₈ denotes H, C₁-C₆-alkyl, phenyl, phenyl-(C₁-C₆-alkyl), COR₁₀, COOR₁₀, CHO, CONH₂, CONHR₁₀, SO₂—(C₁-C₆-alkyl), SO₂-phenyl, while the phenyl group may be mono- or di-substituted by Cl, F, CF₃, C₁-C₄-alkyl, OH and/or C₁-C₄-alkoxy, and preferably denotes C₁-C₄-alkyl, particularly isopropyl;
R₉ denotes H or C₁-C₆-alkyl, preferably H or C₁-C₄-alkyl, particularly isopropyl; or
R₈ and R₉ taken together represent a C₄-C₆-alkylene group;
R₁₀ denotes C₁-C₆-alkyl, C₅-C₇-cycloalkyl, aryl, heteroaryl, aralkyl, or heteroaryl-(C₁-C₆-alkyl), preferably C₁-C₄-alkyl,
   while the aryl groups mentioned under groups R₂ and R₁₀ denote phenyl or naphthyl, the heteroaryl groups denote pyrrole, pyrazole, imidazole, furanyl, thienyl, pyridine, or pyrimidine and in each case may be mono- or polysubstituted by Cl, F, CF₃, C₁-C₄-alkyl, OH, HSO₃—O, or C₁-C₄-alkoxy, preferably by OH or HSO₃—O—.

The compounds according to formula I have extremely low solubility in water and solubility in the physiological pH range (approx. <0.5 μg/ml) combined with poor wettability. In view of the importance of the LTB₄-antagonists mentioned above there is therefore a constant need to discover ways of improving the bioavailability and hence efficacy of these compounds. Thus, WO 03/007922 describes how the bioavailability of the active substance may be increased if the active substance is formulated together with a wetting agent.

Consequently, an underlying aim of the present invention is to provide a formulation with improved bioavailability for LTB₄-antagonists, i.e. to develop a formulation which releases an active substance of formula I relatively rapidly and completely and thus leads to an increased bioavailability of this active substance. In addition, an orally administered pharmaceutical formulation is to be provided. A further aim of the present invention is to prepare a formulation which is easily handled during the manufacturing process and thereby allows industrial production in reproducible manner while maintaining a consistently high quality.

### BRIEF DESCRIPTION OF THE DRAWINGS

FIG. 1 shows the average plasma concentration of a glucuronide metabolite, on a linear scale, as recorded over 24 hours, after a single dose of the LTB₄ antagonist of formula IA in the form of the base, either as an embedding melt or in the form of a tablet under fasting conditions (parallel groups), according to Example 2;

FIG. 2 shows the average plasma concentration of the glucuronide metabolite, on a logarithmic scale, as recorded over 24 hours, after a single dose of the LTB₄ antagonist of formula IA in the form of the base, either as an embedding melt or in the form of a tablet under fasting conditions (parallel groups), according to Example 2;

FIG. 3 shows the average plasma concentration of the glucuronide metabolite, on a linear scale, as recorded over 72 hours, after a single dose of the LTB₄ antagonist of formula IA in the form of the base, either as an embedding melt or in the form of a tablet under fasting conditions (parallel groups), according to Example 2;

FIG. 4 shows the average plasma concentration of the glucuronide metabolite, on a logarithmic scale, as recorded over 72 hours, after a single dose of the LTB₄ antagonist of formula IA in the form of the base, either as an embedding melt or in the form of a tablet under fasting conditions (parallel groups), according to Example 2;

FIG. 5 shows the results of a release test carried out with a suspension containing a ground powder of a pharmaceutical formulation according to Example 3a containing 10% of an LTB₄-antagonist.

FIG. 6 shows the results of a release test carried out with a suspension containing a ground powder of a pharmaceutical formulation according to Example 3b containing 5% of an LTB₄-antagonist.

FIG. 7 shows the results of a release test carried out with a suspension containing a granulated pharmaceutical formulation according to Example 3c containing 10% of an LTB₄-antagonist.

FIG. 8 shows the results of Example 3d, which is a release test carried out with a suspension containing a granulated pharmaceutical formulation according to the present invention containing 15% of an LTB₄-antagonist.

FIG. 9 shows the plasma concentrations of the glucuronide metabolite zwitterion, standardized to a dose of 1 mg/kg, after oral administration to minipigs of various formulations of the LTB₄ antagonist of formula IA in the form of the base according ot Example 5; and

FIG. 10 shows the dose-standardized Cₘₐₓ and AUC₀₋₂₄h values of the glucuronide metabolite zwitterion after oral administration to minipigs of various pharmaceutical formulations of the LTB₄ antagonist of formula IA in the form of the base according to Example 5.

### DETAILED DESCRIPTION OF THE INVENTION

The problem set out above is solved by the features of claim 1. This provides a pharmaceutical formulation containing an LTB₄-antagonist of formula 1, the pharmacologically acceptable acid addition salt, glycoside, O-sulphate, or glucuronide thereof as active substance as well as optionally at least one pharmacologically acceptable excipient and/or carrier, while the active substance is in the form of a solid solution or solid dispersion in a polymer matrix.

The active substance of formula I may be present in the pharmaceutical formulation according to the invention in the form of a physiologically acceptable acid addition salt, for example. By physiologically acceptable acid addition salts are meant, according to the invention, pharmacologically acceptable salts which are selected from the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid. If desired, mixtures of the above-mentioned acids may also be used to prepare the salts. According to the invention, the salts of formula I selected from the group consisting of hydrochloride, hydrobromide, sulphate, phosphate, fumarate, and methanesulphonate are preferred. The salts are particularly preferably selected from the hydrochloride, hydrobromide, and fumarate. The active substance may optionally be present in the form of a hydrate. However, according to the invention, the compound of formula I is preferably in the form of the free base.

A particularly preferred compound of formula I is the compound amelubant, i.e. [4-((3-((4-(1-(4-hydroxyphenyl)-1-methylethyl)phenoxy)methyl)benzyl)oxy)benzene-carboximidamide-N-ethylcarboxylate], shown below in formula IA:

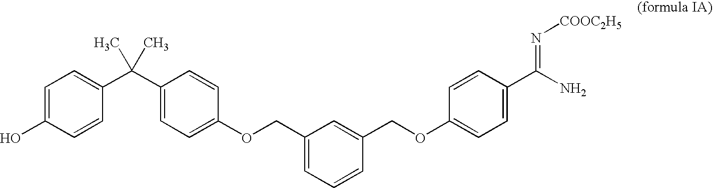

The compounds of formula I wherein R₁ is other than hydrogen are generally prodrugs which are converted in-vivo into the corresponding compounds of formula I wherein R₁ is hydrogen. For example, from the compound IA is formed, in-vivo, the compound of formula IA1: 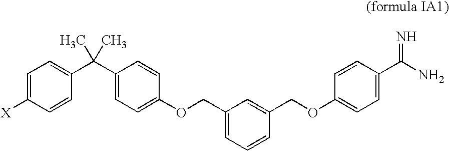
wherein X denotes OH, HSO₃—O, a carbohydrate group of formula C₆H₁₁O₅—O or a glycosyl group and are metabolites of the above compound.

The pharmaceutical formulation according to the invention is a solid solution or dispersion of the active substance in the form of an LTB4-antagonist of formula I, particularly of formula IA, in a matrix of one or more polymers. Polymers or polymer mixtures which are particularly suitable within the scope of the invention are hydrophilic or water-soluble polymers, which help to enable the active substance to be formulated as a solid solution/dispersion. By “water-soluble” in this context are meant not only true solutions but also colloidal solutions of the polymer or polymers in water.

The polymers used may be, for example, polyethyleneglycols, polypropyleneglycols, cellulose ethers, polyvinylpyrrolidones, polyvinyl acetates, copolymers, and mixtures thereof. Particularly preferred polymers are poloxamers, i.e. known copolymers of polyethyleneglycols and polypropyleneglycols, methylcellulose, ethylcellulose, propylcellulose, carboxymethylcellulose, ethylhydroxyethylcellulose, hydroxypropyl-cellulose, N-vinylpyrrolidone (NVP) homopolymers, such as those sold by BASF under the registered trademark Kollidon®, as well as mixed polymers of polyvinylpyrrolidone and polyvinyl acetate or polyethyleneglycols with various chain lengths. Most particularly preferred are poloxamers, such as for example Poloxamer 188.

The pharmaceutical formulation according to the invention optionally contains in addition to the active substance/s and the polymer matrix one or more excipients and/or carriers, such as fillers, binders, disintegrants, break-down agents, flow agents or flow regulators, lubricants, separators, pH correctors, particularly buffers, antioxidants and dyes.

Suitable fillers which have proved particularly advantageous for use within the scope of the present invention are carbohydrates such as lactose or mannose, particularly finely divided lactose, or sugar alcohols such as mannitol, sorbitol, or xylitol, particularly mannitol.

Binders which are preferred according to the invention are selected from the group consisting of: powdered cellulose, microcrystalline cellulose, sorbitol, starch, polyvinylpyrrolidone (povidone), copolymers of vinylpyrrolidone with other vinyl derivatives (copovidone), cellulose derivatives, particularly methylhydroxypropyl cellulose, e.g. Methocel® A15LV from Dow Chemical Company, and mixtures of these compounds. Preferably, powdered cellulose, particularly microcrystalline cellulose and/or copovidone are present as binders.

The pharmaceutical formulation according to the invention may also contain, in addition to the above-mentioned constituents, breakdown agents, which are sometimes also referred to as disintegrants. These are preferably selected according to the invention from the group consisting of sodium starch glycolate, cross-linked polyvinylpyrrolidones (crospovidone), croscarmellose-sodium salt (cellulose-carboxymethylether sodium salt, cross-linked), sodium carboxymethylcellulose, dried maize starch, and mixtures thereof. It is particularly preferred within the scope of the present invention to use sodium starch glycolate, crospovidone and preferably crospovidone or croscarmellose sodium salt.

The pharmaceutical formulation according to the invention may contain as further constituents flow agents or flow regulators as well as lubricants. These include within the scope of the present invention for example silicon dioxide, talc, stearic acid, sodium stearylfumarate, magnesium stearate, and glyceroltribehenate. Preferably, magnesium stearate is used according to the invention.

In addition, the pharmaceutical formulation according to the invention may contain one or more synthetic or natural pharmaceutically acceptable colorings, preferably indigo carmine.

Naturally, other excipients and carriers known to the skilled man may be contained in the pharmaceutical formulation according to the invention. The proportion of excipients and/or carriers, based on the total mass of the formulation, is preferably in the range from about 50 to about 99.5 wt.-%, particularly about 90 to about 99 wt.-%.

Based on the total mass of the pharmaceutical formulation according to the invention the compound of formula I, for example of formula IA, according to the invention is preferably present in an amount of up to about 0.5 to about 50 wt.-%, particularly preferably about 0.5 to about 25 wt.-%, particularly about 1 to about 10 wt.-%. Preferably the proportion of free base, based on the total mass of the formulation, is between about 0.5 and about 25 wt.-%, particularly preferably between about 1 and about 10 wt.-%.

Preferably, the active substance is used in crystalline, unground form or in ground form, particularly in jet-ground form.

Without being tied to a particular theory, it is assumed that the pharmaceutical formulation according to the invention may act as follows:

In the new galenic formulation in the form of solid solutions/dispersions provided according to the invention, the polymer matrix initially dissolves after administration when using water-soluble polymers, where “water-soluble” is defined as above. This takes place in the aqueous medium, i.e. in the gastrointestinal tract, leaving behind the active substance which is dissolved or finely divided in the polymer matrix. This dissolves partly in parallel with the dissolution of the polymer matrix and partly afterwards, producing a supersaturated solution. The dissolved concentration of the active substance obtained is higher than is theoretically possible and even achievable in a thermodynamically stable form. Nevertheless, it is surprisingly possible to produce supersaturation. This can be assisted by a suitable choice of excipients and polymers used. Thus, with the invention, the supersaturated solutions/dispersions are stabilized for different lengths of time to the desired extent. Thus, the high concentration of dissolved active substance leads to increased absorption of the active substance in vivo, i.e. the active substance is more available to an organism and can thus develop its activity to a significantly greater extent.

This supersaturation of the active substance concentration achieved according to the invention by the specially developed galenic formulation has been demonstrated by release tests in vitro and by bioavailability studies in vivo, as is shown in detail in the Examples.

The invention also relates to a process for preparing the pharmaceutical formulation described above comprising the steps of:
(1) melting a polymer or a mixture of polymers;
   (2) dissolving or dispersing an active substance selected from the LTB₄ antagonists of formula I as defined above in the melt;
(3a) pouring the melt into suitable moulds and leaving the melt to harden while cooling or
(3b) leaving the solid solution or solid dispersion obtained to harden while cooling and then comminuting the solid solution or solid dispersion obtained into suitable shapes.

First, in step (1) according to the invention a melt of one or more polymers is produced. These are the polymers described above in detail. This is followed in step (2) by the dissolving or dispersing of the LTB₄ antagonist active substance of formula I as defined above, e.g. of formula IA, in the molten polymer or polymers. It is particular preferred to use the active substance in step (2) in crystalline, unground form or in ground form particularly in jet-ground form. In addition, it has been found particularly useful to screen the active substance after grinding. Preferably the active substance used then has a mean particle size (D 50) of about 1 μm to about 7 μm, particularly about 1.5 μm to about 3 μm. This was determined by the laser diffractometry method (e.g. Sympatec apparatus using HELOS software, RODOS dry disperser).

As soon as the active substance or substances are dissolved or dispersed in the melt, one of two alternative procedures can be followed. Either the melt is poured into suitable moulds and left to harden as they cool (step 3a) or the solid solution or solid dispersion obtained is left to go cold and then cut up into the required shapes (step 3b). Preferably, this is done by grinding but any known technique may be used. Then another screening may be carried out.

After cooling, so-called solid solutions are formed, in which the active substance is present in the hardened polymer matrix in molecular dispersion. If the active substance re-crystallizes during cooling or does not totally dissolve in the melt, so-called solid dispersions are formed. The products obtained in step (3a) or (3b) may expediently be processed into tablets, film-coated tablets, sugar-coated tablets, powders, or sachets of powder, or may be packed directly into capsules, such as hard gelatin capsules, for example.

The invention also relates to a solid solution or solid dispersion containing an LTB₄ antagonist of formula I as hereinbefore defined, in a polymer matrix.

The invention also relates to a solid solution or solid dispersion containing an LTB₄ antagonist of formula IA as hereinbefore defined, in a polymer matrix.

Another embodiment of the present invention for preparing the pharmaceutical formulation or the solid solutions or dispersions is preparation using a melt extrusion process, which is also known per se in the art and requires no further explanation.

The invention also relates to the use of the pharmaceutical formulation for preparing a pharmaceutical preparation with increased bioavailability for treating or preventing illnesses in which LTB₄ antagonists may be used therapeutically or preventively.

In particular, the invention also relates to the use of the pharmaceutical formulation according to the invention for preparing a pharmaceutical preparation for the treatment or prevention of arthritis, asthma, chronic obstructive pulmonary diseases, psoriasis, ulcerative colitis, Alzheimer's disease, shock, reperfusion injury/ischaemia, cystic fibrosis, arteriosclerosis, and multiple sclerosis.

The advantages associated with the present invention are numerous: The invention provides a new galenic formulation which is a solid solution or solid dispersion of an LTB₄ antagonist as active substance in a polymer matrix. This results in an extraordinary improvement in the dissolving characteristics and hence the bioavailability of the active substance, which yields an active substance concentration which is inherently thermodynamically unstable and therefore supersaturated. Nevertheless, the teaching according to the invention makes it possible to prepare a physically and chemically stable formulation. Surprisingly, therefore, it is possible to stabilize supersaturated solutions/dispersions with a high content of active substance, as a result of which the active substance, when released, is more readily available to the body and can develop its activity to a significantly higher degree.

This is demonstrated not only by in vitro release tests; it has also been found in tests carried out in vivo as shown, for example, by the bioavailability study and the release tests on mini-pigs in the Examples.

The exceedingly favorable stability of the pharmaceutical formulation according to the invention and the excellent bioavailability in humans and animals was unexpected and also unforeseeable.

The Examples that follow serve to illustrate the formulations according to the invention. They are intended solely as possible methods described by way of example without restricting the invention to their contents.

### EXAMPLES

In the Examples that follow, the pharmaceutical formulation according to the invention is referred to simply as the “embedding melt” or “Embedding Polymer Melt” and abbreviated to “EPM.”

### Example 1

Preparation of a embedding melt according to the invention containing a 1% charge of active substance

Active substance: LTB₄ antagonist of formula IA in the form of the base, amount: 75 mg polymer matrix amount: 7.5 g (1% embedding melt)

I. Composition

Ingredients

| | |
|---|---|
| (01) LTB₄ antagonist of formula IA, base form, jet-ground | 75 mg* |
| (02) Poloxamer 188 Pharm | 7425 mg |
| Total | 7500 mg |

*The content of active substance must be determined before weighing

II. Product Description

Granules/Powder

| | |
|---|---|
| Appearance: | fine, white, waxy powder |
| Particle size: | ≦500 μm |
| Color: | white |
| Fill volume: | 7.5 g |

III. Manufacturing Process
1 Batch=606 g

1. Melting the Poloxamer 188 Pharm

650,000 g of Poloxamer 188 Pharm (02) are melted at 80° C. in a crystallizing dish of Ø190 mm in a vacuum drying cupboard at an absolute pressure of 100 to 200 mbar over a period of 4 hours.

2. Screening the Active Substance

6.0606 g of the LTB₄ antagonist of formula IA in the form of the base (01) are jet-ground and screened in an analytical screen with a mesh size of 800 μm.

3. Preparing the Embedding Melt

The laboratory reactor is preheated for approx. 30 min at a water bath temperature of 90° C. The laboratory reactor is filled with 600,0000 g of Poloxamer 188 Pharm (02) (liquid). The anchor stirrer is set to 20 rpm, the direction of rotation is set to the right and an absolute pressure of 100 to 200 mbar is applied. After 5 min., the reactor is opened and, within 5 min., all the LTB₄ antagonist of formula IA in the form of the base (01) in jet-ground form is added to the laboratory reactor in one go (6.0606 g) and the reactor is closed. The anchor stirrer is set to 20 rpm and again the direction of rotation is set to the right. 3 min after the addition of active substance the absolute pressure is set at 100 to 200 mbar. 5 min after the addition of active substance the anchor stirrer speed is increased to 100 rpm. 10 min after the addition of active substance the anchor stirrer is set to rotate to the left. From the 15th to the 20th minute the laboratory reactor is opened and any residues of active substance on the anchor stirrer, temperature sensor and glass wall are wiped off and returned to the melt. The laboratory reactor is closed again, an absolute pressure of 100 to 200 mbar is applied, the speed is left at 100 rpm, and the anchor stirrer is set to rotate to the right. 20 min after the addition of active substance the temperature setting of the water bath is returned to 86° C. 25 min after the addition of active substance the anchor stirrer is set to rotate to the left. Then, 35 min after the addition of active substance, the anchor stirrer is set to rotate to the right again. 40 min after the addition of active substance the anchor stirrer speed is set to 20 rpm. 60 min after the addition of active substance the laboratory reactor is opened and the embedding melt is poured out in a thin layer on a sheet of glass or stainless steel.

Process/Equipment Data:

| | |
|---|---|
| Laboratory reactor: | IKA Laboratory reactor system LR-A 1000 |
| Stirrer: | IKA stirrer |
| drive: | RE 162 A |
| control: | RE 162 P Analog |
| Circulating thermostat: | mgw Lauda C3 |
| type: | T 1 |

4. Hardening

The embedding melt is poured out in a thin layer on a sheet of glass or stainless steel (layer thickness approx. 1.5 to 2.5 mm) and left to harden. The hardening time is approx. 2 to 3 h. The hardened embedding melt is scraped off from the sheet of glass or stainless steel using a dough scraper and stored in a brown wide-necked flask.

5. Grinding and Screening

The individual flakes are ground up using a water-cooled IKA Universal Mill and the ground material is screened using a 500 μm Kressner screen. The grinding and screening process is repeated until all the embedding melt has been ground to ≦500 μm.

Process/Equipment Data:

| | |
|---|---|
| Mill: | IKA-cross-beater/universal mill |
| Type: | M 20 |
| Grinding time: | 3 × approx. 5 sec/filling |

6. Filling

Under GMP conditions (Good Manufacturing Practice) the granulated material is packed into the sterile-packaged glass flasks and the pilfer-proof closure is sealed using a PfP flanging machine.

| | |
|---|---|
| Contents: | 7.500 g |
| Tolerance during filling | 7.470 g to 7.530 g |

IV. In-Process Controls

1. Liquid Embedding Melt

About 30 to 35 min after the addition of active substance it is advantageous if the active substance is dissolved. From this time onwards, the embedding melt is clear. No undissolved flakes should be visible in the embedding melt shortly before the emptying of the laboratory reactor.

2. Solid Embedding Melt

As the embedding melt hardens its appearance changes from a clear liquid to a waxy white substance.

3. Granules/Powder

Fine particles, white, particle size ≦500 μm.

The correct particle size is achieved when everything passes through the 500 μm screen.

4. Fill quantity

The masses packed are between 7.470 g and 7.530 g.

### Example 2

The embedding melt prepared in Example 1 containing a 1% charge of the active substance (75 mg of the LTB₄ antagonist of formula IA in the form of the base) was tested on humans. The control used was a tablet which also contained 75 mg of the LTB₄ antagonist of formula IA in the form of the base. The plasma concentration of the glucuronide metabolite was determined. The glucuronide metabolite is the LTB₄ antagonist of formula IA in the form of the base, whose structure was explained in Example 1, except that the ethylcarboxyl group has been cleaved (═N—CO₂C₂H₅→═NH) and at the same time the hydroxy group has been glycosylated at the “left-hand” phenyl ring (LTB₄ antagonist of formula IA1). The glucuronide metabolite is present as a zwitterion. The LTB₄ antagonist of formula IA in the form of the base is converted into the glucuronide metabolite in the human body, as explained, and constitutes an active metabolite thereof.

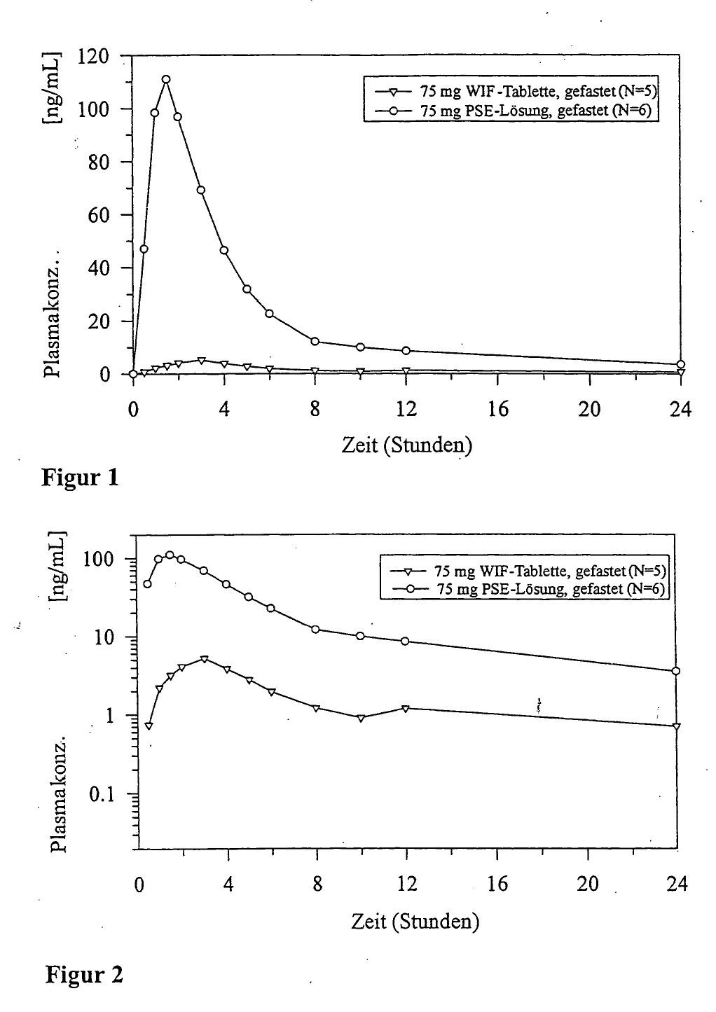

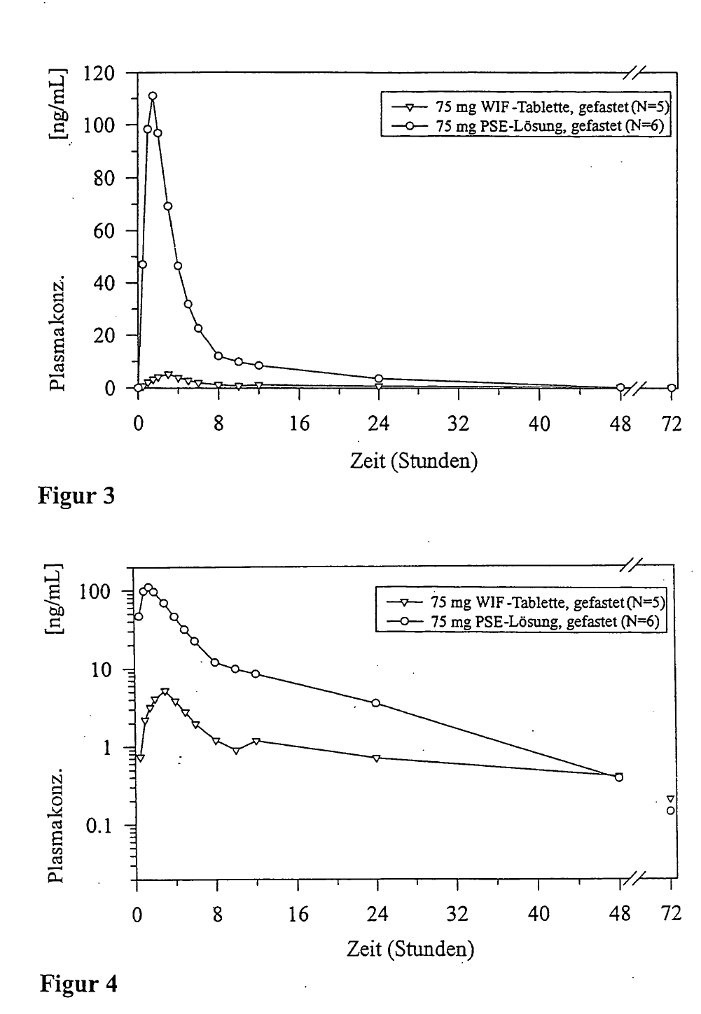

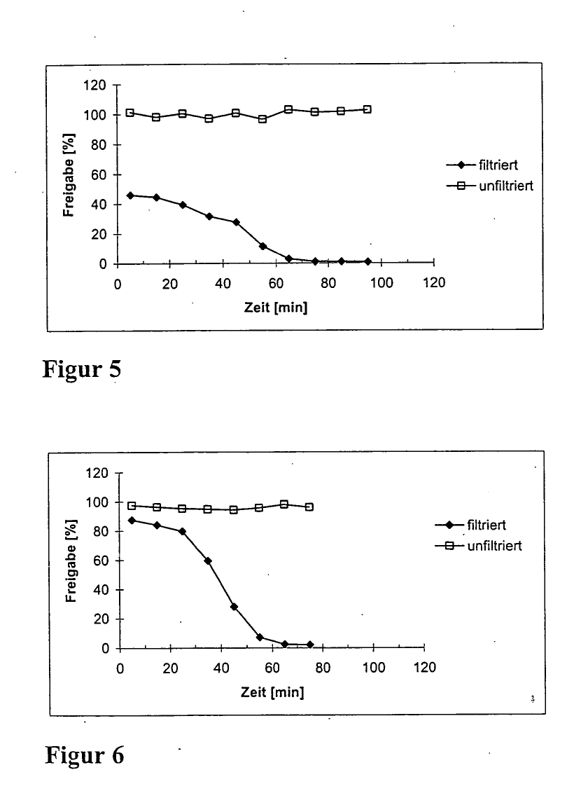

The results are shown in FIGS. 1 to 4. FIGS. 1 to 4 show the average plasma concentration of the glucuronide metabolite, recorded against time, after a single dose of 75 mg of the LTB₄ antagonist of formula IA in the form of the base, either as a embedding melt according to the invention (EPM) or in the form of a WIF tablet (wettability improved formulation, a formulation corresponding to the prior art according to WO 03/007922, containing a wetting agent)—under fasting conditions (parallel groups), at different times in each case.

The tests on humans clearly show that the plasma concentration of the formulation according to the invention exceeds that of a conventional tablet with the same active substance in the same dosage, i.e. the quantity of active substance available is many times greater with the formulation according to the invention than with a conventional tablet.

### Example 3

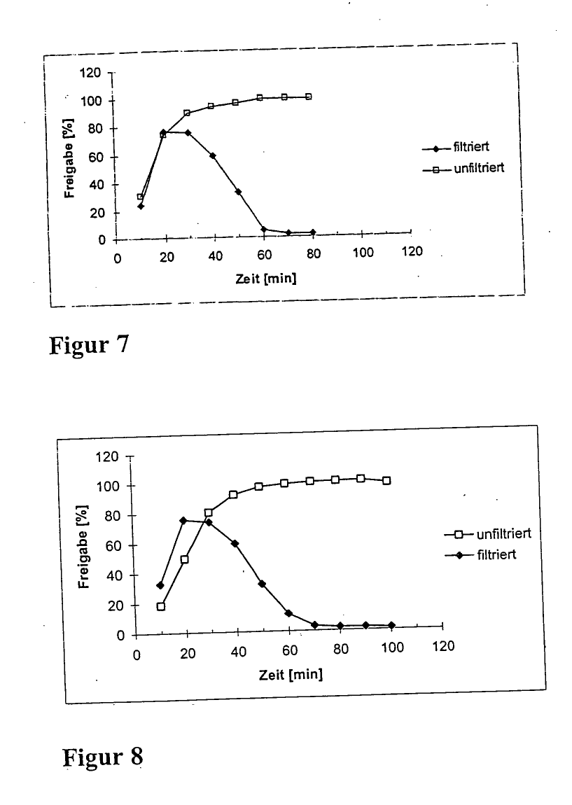

Release results of embedding melts containing different loads of active substance Examples 3a) and 3b) that follow relate to a suspension of the EPM powder in water. Release tests were then carried out with this suspension. The results are shown in FIGS. 5 and 6. On the one hand, the amount of the LTB₄ antagonist of formula IA in the form of the base released (“unfiltered”) and, on the other hand, the colloidally dissolved fraction with particle sizes of less than 220 nm, which are marked as “filtered” in FIGS. 5 and 6 were evaluated.

On the one hand, the total amount of active substance released by the pharmaceutical preparation was shown. The release curves normally used for characterizing solid oral preparations were used for this.

On the other hand, the pharmaceutical fractions which cannot be filtered off using a 0.22 μm filter are also shown. Their particle size is therefore less than 220 nm. Thus, the active substance is dissolved or at least colloidally dissolved, i.e. very finely divided, even if these concentrations are far higher than the saturation concentrations of the active substance. This supersaturation is maintained for a certain length of time before the active substance crystallizes and can therefore be removed by filtration, i.e. has a particle size >220 nm. The other Examples 3c) and 3d) that follow relate to release tests on EPM powder packed into hard gelatin capsules. Here again, both the total quantity of the LTB₄ antagonist of formula IA in the form of the base released and also the colloidally dissolved fraction were investigated.

3a) EPM Ground—10% Active Substance Charge

(suspended)
   Composition EPM 10%:
   67.41 g of the LTB₄ antagonist of formula IA in the form of the base
   600 g Poloxamer 188 Pharm (polymer matrix)
   Preparation: In the beaker
   Release of 499.8 mg of EPM, particle size <0.5 mm, suspended in 5 mL of de-ionized water, added after 5 minutes to the release medium
   Release medium: 500 mL of 0.1 N HCl with 50 mg of Methocel A 15 LV

The results are shown in FIG. 5.

3b) EPM Ground—5% Active Substance Charge

(suspended)
   Composition EPM 5%:
   31.9369 g the LTB₄ antagonist of formula IA in the form of the base 600.09 g Poloxamer 188 Pharm
   Preparation: In the laboratory reactor
   Release of 1000.2 mg of EPM, particle size <0.5 mm, suspended in 5 mL of de-ionized water, added after 5 minutes to the release medium
   Release medium: 500 mL of 0.1 N HCl with 50 mg of Methocel® A 15 LV

The results are shown in FIG. 6.

3c) Capsule Containing Granulated EPM—10% Active Substance Charge

Composition EPM 10%:
1002.4 mg the LTB₄ antagonist of formula IA in the form of the base 9021.8 mg Poloxamer 188 Pharm
Capsule filled with 300 mg EPM, particle size 0.5-0.8 mm
Release medium: 400 mL of 0.1 N HCl with 20 mg Methocel® A15LV

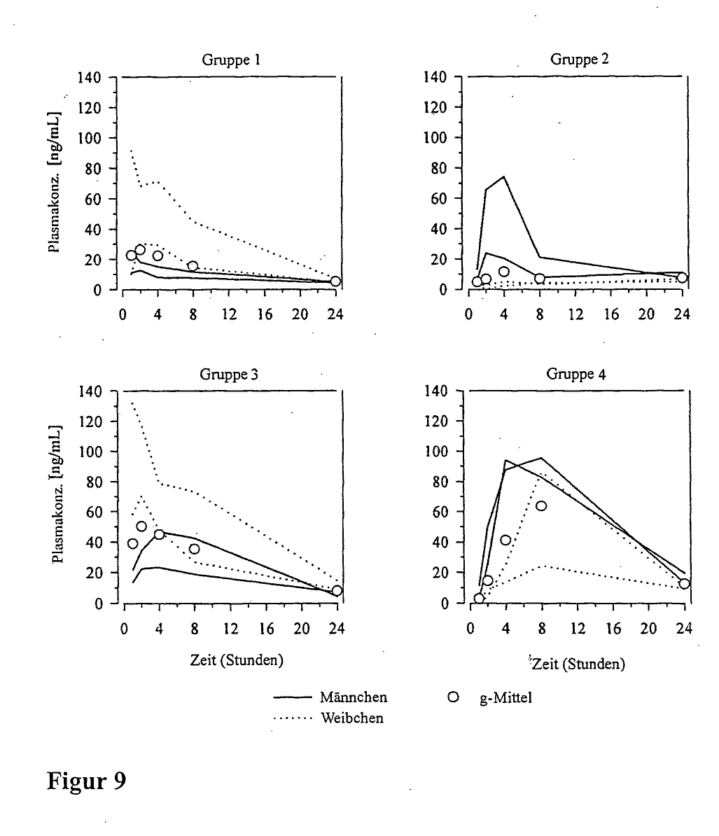

The results are shown in FIG. 7.

3d) Capsule with EPM Granules—15% Active Substance Charge

Composition EPM 15%:
4.5037 g the LTB₄ antagonist of formula IA in the form of the base 25.5067 g Poloxamer 188 Pharm
Capsule filled with 332.7 mg of EPM, particle size <0.5 mm
Release medium: 500 mL of 0.1 N HCl with 50 mg Methocel® A15LV

The results are shown in FIG. 8.

### Example 4

Preparation of an embedding melt according to the invention with a 10% active substance charge:

active substance: the LTB₄ antagonist of formula IA in the form of the base, amount: 75 mg amount of polymer matrix: 0.750 g (10% embedding melt)

I. Composition

Ingredients

| | |
|---|---|
| (01) The LTB₄ antagonist of formula IA | 75 mg* |
| base form, jet-ground 98.9% | |
| (02) Poloxamer 188 Pharm | 675 mg |
| TOTAL | 750 mg |

*The active substance content must be determined before weighing out

II. Product Description

Granules/Powder

| | |
|---|---|
| Appearance: | fine, white, slightly waxy powder |
| Particle size: | ≦500 μm |
| Color: | White |
| Fill volume: | 750 mg |

III. Production Method
1 Batch=667 g=89 Bottles

1. Melt the Poloxamer 188 Pharm

As the melting poloxamer in the laboratory reactor would overload the anchor stirrer, the poloxamer is melted separately. In a crystallizing dish of Ø 190 mm 650,000 g Poloxamer 188 Pharm (02) are melted at 80° C. in a vacuum drying cupboard at an absolute pressure of 100 to 200 mbar over a period of 4 hours.

2. Screen the Active Substance

66.6667 g of the LTB₄ antagonist of formula IA in the form of the base (01) are jet-ground and screened in an analytical screen with a mesh size of 800 μm.

3. Prepare the Embedding Melt

The laboratory reactor is preheated for approx. 30 min at a water bath temperature of 90° C. The laboratory reactor is filled with 600,0000 g of Poloxamer 188 Pharm (02) (liquid). The anchor stirrer is set to 20 rpm, the direction of rotation is set to the right, and an absolute pressure of 100 to 200 mbar is applied. After 5 min., the reactor is opened and, within 5 min., all the LTB₄ antagonist of formula IA in the form of the base in jet-ground form is added to the laboratory reactor in one go (66.6667 g), and the reactor is closed. The anchor stirrer is set to 20 rpm and again the direction of rotation is set to the right. 3 min after the addition of active substance the absolute pressure is set at 100 to 200 mbar. 5 min after the addition of active substance the anchor stirrer speed is increased to 100 rpm. 10 min after the addition of active substance the anchor stirrer is set to rotate to the left. From the 15th to the 20th minute the laboratory reactor is opened and any residues of active substance on the anchor stirrer, temperature sensor and glass wall are wiped off and returned to the melt. The laboratory reactor is closed, an absolute pressure of 100 to 200 mbar is applied, the speed is left at 100 rpm, and the anchor stirrer is set to rotate to the right. 25 min after the addition of active substance the anchor stirrer is set to rotate to the left. 35 min after the addition of active substance, the water bath temperature setting is returned to 86° C. and the direction of rotation of the anchor stirrer is set to the right again. 40 min after the addition of active substance the anchor stirrer speed is set to 20 rpm. 60 min after the addition of active substance the laboratory reactor is opened and the embedding melt is poured out in a thin layer on a sheet of glass or stainless steel.

Process/Equipment Data (as in Example 1)

4. Hardening

The embedding melt is poured out in a thin layer on a sheet of glass or stainless steel (layer thickness approx. 1.5 to 2.5 mm) and left to harden. The hardening time is approx. 2 to 3 h. The hardened embedding melt is scraped off from the sheet of glass or stainless steel using a dough scraper and stored in a brown wide-necked flask.

5. Grinding and screening

The individual flakes are ground up using a water-cooled IKA Universal Mill and the ground material is screened using a 500 μm Kressner screen. The grinding and screening process is repeated until all the embedding melt has been ground to ≦500 μm.

Process/Equipment Data (as in Example 1)

6. Filling

Under GMP conditions, the granulated material is packed into the sterile-packaged glass flasks and the pilfer-proof closure is sealed using a PfP flanging machine.
Contents: 750 mg
Tolerance during filling 745 mg to 755 mg
   IV. In-Process Controls (see Example 1)

### Example 5

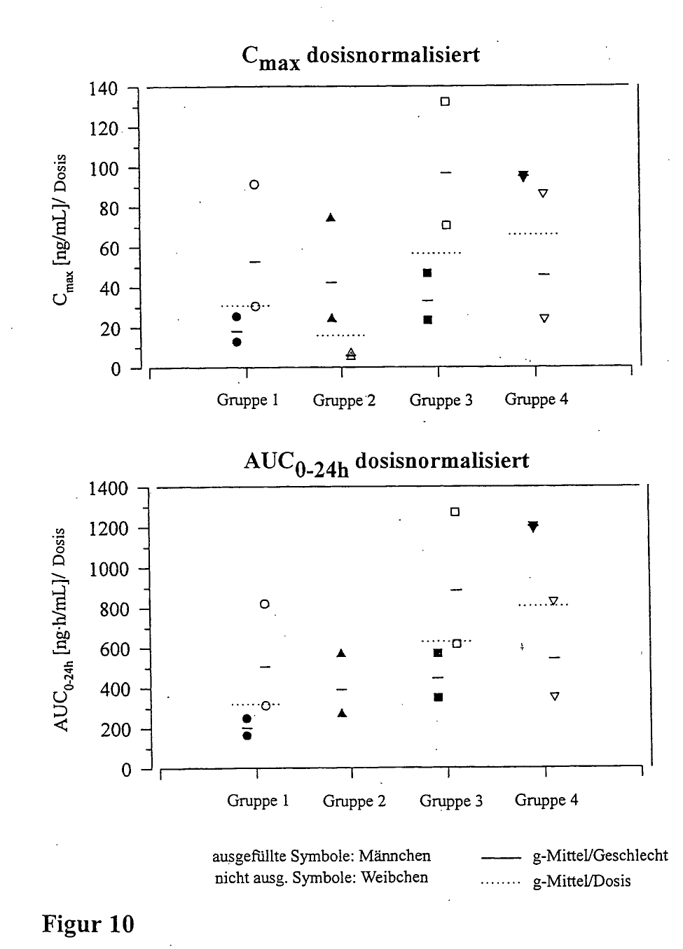

The embedding melts according to the invention containing 5 and 10% active substance charge, as prepared in Example 4, were tested on minipigs. A bioavailability study was carried out, the results of which are shown in FIGS. 9 and 10.

a) Summary

A relative bioavailability study with different formulations of the LTB₄ antagonist of formula IA in the form of the base (5% EPM, 10% EPM, and tablet) was carried out after oral administration to minipigs. Two EPM formulations, one with a 5% active substance charge, and one with a 10% active substance charge of the LTB₄ antagonist of formula IA in the form of the base, were examined.

b) Objective

The aim is to study the absorption of various pharmaceutical formulations of the LTB₄ antagonist of formula IA in the form of the base on minipigs.

c) Method

c1) Animal Tests

Two EPM formulations were used, one containing 5% of the LTB₄ antagonist of formula IA in the form of the base and one containing 10% of the LTB₄ antagonist of formula IA in the form of the base. The EPMs were stored in glass containers and suspended in 50 ml of tap water immediately before use. After the dose had been administered, the flasks were washed once with another 50 ml of tap water which was also given to the animals.

6 tablets containing 75 mg of the LTB₄ antagonist of formula IA in the form of the base were each placed in a capsule (size 000). After the capsules had been administered, the container was rinsed with 50 ml of tap water. The food was provided 3 to 4 hours after administration, except in the case of group 2, where the food was given 15 minutes after administration.

TABLE 1

| group | formulation | dose [mg] | sex | subject | weight [kg] |
|---|---|---|---|---|---|
| 1 | EPM 10% | 300 | M | 53918 | 19.3 |
| | | 300 | M | 53879 | 31.5 |
| | | 300 | F | 53825 | 36.0 |
| | | 300 | F | 53837 | 24.0 |
| 2 | Tablet in capsule/food | 1350 | M | 51853 | 27.3 |
| | 15 min after taking | 900 | M | 51726 | 28.4 |
| | | 1350 | F | 51826 | 29.9 |
| | | 900 | F | 51830 | 34.1 |
| 3 | EPM 5% | 300 | M | 53918 | 20.1 |
| | | 300 | M | 53879 | 31.6 |
| | | 300 | F | 53825 | 35.4 |
| | | 300 | F | 53837 | 24.9 |
| 4 | Tablet/food | 1350 | M | 51853 | 30.1 |
| | 4 h after taking | 1350 | M | 51726 | 31.9 |
| | | 1350 | F | 51826 | 30.0 |
| | | 1350 | F | 51830 | 33.5 |

M . . . male
F . . . female

c2) Bioanalytical Parameters

The plasma concentrations of glucuronide metabolite in zwitterion form were quantified by HPLC-MS/MS.

d) Results

The dose-standardized concentrations of the glucuronide metabolite zwitterion, the individual and average dose-standardized AUC_{O-24}h and Cₘₐₓ values and the tₘₐₓ values are shown in section f). In addition, the pharmacokinetic parameters are shown in Table 2.

The results are also shown in FIGS. 9 and 10. FIG. 9 shows the plasma concentrations of the glucuronide metabolite zwitterion, standardized to a dose of 1 mg/kg, after oral administration to minipigs of various formulations of the LTB₄ antagonist of formula IA in the form of the base. FIG. 10 shows the dose-standardized Cₘₐₓ and AUC₀₋₂₄ₕ values of the glucuronide metabolite zwitterion after oral administration to minipigs of various pharmaceutical formulations of the LTB₄ antagonist of formula IA in the form of the base.

TABLE 2
Summary of the pharmacokinetic parameters

| | | AUC_{0-24 h} | | | Cₘₐₓ | | | tₘₐₓ | |
|---|---|---|---|---|---|---|---|---|---|
| | | | g mean | gCV | | g mean | gCV | mean | range |
| group | formulation | N | [ng · h/ml]/dose | [%] | N | [ng/ml]/dose | [%] | [h] | [h] |
| 1 | EPM (10%) | 4 | 320 | 78 | 4 | 31 | 98 | 1.5 | 1-2 |
| 3 | EPM (5%) | 4 | 630 | 57 | 4 | 56 | 84 | 3 | 1-4 |
| 2 | tablet + food | 2 | 390 | 57 | 4 | 16 | 190 | 14 | 2-24 |
| 4 | tablet | 4 | 807 | 63 | 4 | 66 | 75 | 8 | 4-8 |

N . . . number of experimental animals
AUC_{0-24 h} . . . Area under the curve from 0 to 24 hours
Cₘₐₓ . . . maximum plasma concentration
tₘₐₓ . . . time to reach maximum plasma level
g mean . . . geometric mean value
g CV . . . geometric standard deviation
mean . . . mean value

Variability: The inter-individual variability of the plasma concentrations was high. Differences in release were observed between male and female minipigs. However, the differences between the formulation groups were not uniform.

EPM: The dose-standardized AUC₀₋₂₄h and Cₘₐₓ values of the EPM formulations which contained 5% the LTB₄ antagonist of formula IA in the form of the base were about twice as great as the corresponding values of the EPM formulations which contained 10% the LTB₄ antagonist of formula IA in the form of the base.

Tablet: Oral administration of the tablets also led to a substantial release in the animals. It should be pointed out that this formulation was tested on different animals from the EPM. A direct comparison of the results might possibly be misleading on account of the high inter-individual variability. However, the AUC_{O-24}h and Cₘₐₓ values were comparable with the 5% EPM values. Tₘₐₓ was significantly delayed compared with the EPM formulations.

a) Conclusions

The differences in release between male and female minipigs were not consistent between the different formulation groups and are therefore due to inter-individual variability rather than actual gender differences.

It was shown that the ratio of the LTB₄ antagonist of formula IA in the form of the base to Pluronics (=Poloxamer, polymer matrix) affects the release of the glucuronide metabolite zwitterion in the animals. A lower charging of the EPM with the active substance the LTB₄ antagonist of formula IA in the form of the base, i.e. a higher amount of Pluronics (polymer matrix), led to higher the glucuronide metabolite zwitterion plasma concentrations.

With a more conventional formulation, i.e., a tablet of the LTB₄ antagonist of formula IA in the form of the base, systemic release was also achieved in the animals, which was comparable with the release using EPM with a 5% charge of active substance.

f) Summary of the Values Measured

TABLE 3
Dose-standardized plasma concentration of the glucuronide metabolite
zwitterion, after oral administration of 10 mg/kg or 1350 mg (tablet)/minipig, LTB4
antagonist of formula IA in the form of the base
group 1, dose-standardized

| time | 53918 | 53879 | 53825 | 53837 | | mean | | g-mean | |
|---|---|---|---|---|---|---|---|---|---|
| [h] | M | M | F | F | N | [ng/mL] | CV (%) | [ng/mL] | gCV (%) |
| 1 | 11.23 | 25.35 | 91.25 | 10.24 | 4 | 34.52 | 111.4 | 22.71 | 133.7 |
| 2 | 12.69 | 18.17 | 68.17 | 30.40 | 4 | 32.36 | 77.3 | 26.29 | 83.8 |
| 4 | 8.000 | 15.25 | 71.530 | 29.34 | 4 | 31.03 | 91.6 | 22.49 | 118.4 |
| 8 | 7.626 | 11.63 | 44.920 | 14.44 | 4 | 19.65 | 86.9 | 15.49 | 88.1 |
| 24 | 4.285 | 5.068 | 7.002 | 4.899 | 4 | 5.314 | 22.1 | 5.224 | 21.1 |

group 2, dose-standardized

| time | 51853 | 51726 | 51828 | 51830 | | mean | | g-mean | |
|---|---|---|---|---|---|---|---|---|---|
| [h] | M | M | F | F | N | [ng/mL] | CV (%) | [ng/mL] | gCV (%) |
| 1 | 6.822 | 13.45 | 2.500 | 2.500 | 4 | 6.318 | 81.9 | 4.894 | 98.5 |
| 2 | 23.96 | 65.46 | 0.4588 | 2.779 | 4 | 23.16 | 130.0 | 6.687 | 1170.6 |
| 4 | 20.530 | 74.07 | 2.324 | 5.155 | 4 | 25.52 | 130.6 | 11.62 | 305.5 |
| 8 | 7.945 | 21.17 | 4.317 | 3.456 | 4 | 9.222 | 88.9 | 7.078 | 96.4 |
| 24 | 11.150 | 7.599 | 5.049 | 6.822 | 4 | 7.655 | 33.5 | 7.350 | 33.6 |

group 3, dose-standardized

| time | 53918 | 53879 | 53825 | 53837 | | mean | | g-mean | |
|---|---|---|---|---|---|---|---|---|---|
| [h] | M | M | F | F | N | [ng/mL] | CV (%) | [ng/mL] | gCV (%) |
| 1 | 13.81 | 21.75 | 132.1 | 58.30 | 4 | 56.49 | 95.6 | 39.00 | 133.5 |
| 2 | 22.44 | 34.53 | 117.6 | 70.53 | 4 | 61.28 | 69.8 | 50.35 | 84.9 |
| 4 | 23.32 | 46.73 | 78.79 | 47.63 | 4 | 49.12 | 46.3 | 44.97 | 53.3 |
| 8 | 18.85 | 42.54 | 73.33 | 26.90 | 4 | 40.41 | 59.5 | 35.46 | 64.2 |
| 24 | 7.309 | 4.525 | 14.58 | 8.933 | 4 | 8.837 | 48.0 | 8.101 | 51.5 |

group 4, dose-standardized

| time | 51853 | 51726 | 51828 | 51830 | | mean | | g-mean | |
|---|---|---|---|---|---|---|---|---|---|
| [h] | M | M | F | F | N | [ng/mL] | CV (%) | [ng/mL] | gCV (%) |
| 1 | 11.48 | 3.603 | 0.7784 | 2.397 | 4 | 4.565 | 104.1 | 2.964 | 156.1 |
| 2 | 49.76 | 25.41 | 4.153 | 8.504 | 4 | 21.96 | 94.2 | 14.54 | 155.4 |
| 4 | 87.68 | 94.21 | 25.40 | 13.69 | 4 | 55.25 | 75.3 | 41.17 | 120.9 |
| 8 | 95.59 | 82.88 | 86.38 | 24.13 | 4 | 72.25 | 45.0 | 63.75 | 72.6 |
| 24 | 13.20 | 19.71 | 10.50 | 9.372 | 4 | 13.20 | 35.1 | 12.65 | 33.7 |

N . . . number of experimental animals
CV . . . standard deviation (coefficient of variation)
gCV . . . geometric standard deviation
g mean . . . geometric mean value
mean . . . mean value

TABLE 4
Individual and average dose-standardized AUC_{0-24 h} of the glucuronide
metabolite zwitterion, after oral administration to minipigs of different formulations of
the LTB4 antagonist of formula IA in the form of the base.

| | dose | | | AUC_{(0-24 h)}/dose | | mean | CV | g-mean | gCV |
|---|---|---|---|---|---|---|---|---|---|
| group | [mg/kg] | sex | subject | [ng * h/mL] | N | [ng * h/mL] | [%] | [ng * h/mL] | [%] |
| 1 | 15.5 | M | 53918 | 161.9 | | | | | |
| 1 | 9.5 | M | 53879 | 247.4 | 2 | 204.6 | 29.6 | 200.1 | 30.7 |
| 1 | 8.3 | F | 53825 | 819.6 | | | | | |
| 1 | 12.5 | F | 53837 | 310.5 | 2 | 565.1 | 63.7 | 504.4 | 77.6 |
| | | M&F | | | 4 | 384.8 | 77.0 | 317.7 | 77.7 |
| 2 | 49.5 | M | 51853 | 267.5 | | | | | |
| 2 | 31.7 | M | 51726 | 566.6 | 2 | 417.1 | 50.7 | 389.3 | 57.0 |
| 2 | 45.1 | F | 51828 | NA | | | | | |
| 2 | 26.4 | F | 51830 | NA | | | | | |
| | | M&F | | | 2 | 417.1 | 50.7 | 389.3 | 57.0 |
| 3 | 14.9 | M | 53918 | 349.7 | | | | | |
| 3 | 9.7 | M | 53879 | 570.1 | 2 | 459.9 | 33.9 | 446.5 | 35.6 |
| 3 | 8.5 | F | 53825 | 1271 | | | | | |
| 3 | 12 | F | 53837 | 616.1 | 2 | 943.6 | 49.1 | 884.8 | 54.7 |
| | | M&F | | | 4 | 701.7 | 56.6 | 628.6 | 57.2 |
| 4 | 44.9 | M | 51853 | 1206 | | | | | |
| 4 | 42.3 | M | 51726 | 1193 | 2 | 1199.5 | 0.8 | 1199 | 0.8 |
| 4 | 45 | F | 51828 | 832.1 | | | | | |
| 4 | 40.3 | F | 51830 | 354.2 | 2 | 593.1 | 57.0 | 542.9 | 66.3 |
| | | M&F | | | 4 | 896.3 | 44.7 | 807.0 | 62.7 |

TABLE 5
Individual and average dose-standardized Cₘₐₓ of of the glucuronide
metabolite zwitterion, after oral administration to minipigs of different formulations of
the LTB4 antagonist of formula IA in the form of the base.

| | dose | | | C(max)/dose | | mean | CV | g-mean | gCV |
|---|---|---|---|---|---|---|---|---|---|
| group | [mg/kg] | sex | subject | [ng/mL] | N | [ng * h/mL] | [%] | [ng * h/mL] | [%] |
| 1 | 15.5 | M | 53918 | 12.69 | | | | | |
| 1 | 9.5 | M | 53879 | 25.35 | 2 | 19.02 | 47.1 | 17.94 | 52.0 |
| 1 | 8.3 | F | 53825 | 91.25 | | | | | |
| 1 | 12.5 | F | 53837 | 30.40 | 2 | 60.83 | 70.7 | 52.67 | 91.1 |
| | | M&F | | | 4 | 39.92 | 87.7 | 30.74 | 97.5 |
| 2 | 49.5 | M | 51853 | 23.96 | | | | | |
| 2 | 31.7 | M | 51726 | 74.07 | 2 | 49.02 | 72.3 | 42.13 | 94.4 |
| 2 | 45.1 | F | 51828 | 5.049 | | | | | |
| 2 | 26.4 | F | 51830 | 6.822 | 2 | 5.936 | 21.1 | 5.869 | 21.5 |
| | | M&F | | | 4 | 27.48 | 117.2 | 15.72 | 189.3 |
| 3 | 14.9 | M | 53918 | 23.32 | | | | | |
| 3 | 9.7 | M | 53879 | 46.73 | 2 | 35.03 | 47.3 | 33.01 | 52.3 |
| 3 | 8.5 | F | 53825 | 132.1 | | | | | |
| 3 | 12 | F | 53837 | 70.53 | 2 | 101.3 | 43.0 | 96.52 | 46.6 |
| | | M&F | | | 4 | 68.17 | 68.6 | 56.45 | 83.6 |
| 4 | 44.9 | M | 51853 | 95.59 | | | | | |
| 4 | 42.3 | M | 51726 | 94.21 | 2 | 94.90 | 1.0 | 94.90 | 1.0 |
| 4 | 45 | F | 51828 | 86.38 | | | | | |
| 4 | 40.3 | F | 51830 | 24.13 | 2 | 55.26 | 79.7 | 45.65 | 112.0 |
| | | M&F | | | 4 | 75.08 | 45.6 | 65.82 | 75.3 |

TABLE 6
Individual and average dose-standardized tₘₐₓ of of the
glucuronide metabolite zwitterion, after oral administration to
minipigs of different formulations of the LTB4 antagonist
of formula IA in the form of the base.
tₘₐₓ

| | dose | | | mean | range |
|---|---|---|---|---|---|
| group | [mg/kg] | sex | N | [h] | [h] |
| 1 | 9.5-15.5 | M | 2 | 1.5 | 1-2 |
| 1 | 8.3-12.5 | F | 2 | 1.5 | 1-2 |
| 1 | 8.3-15.5 | M&F | 4 | 1.5 | 1-2 |
| 2 | 31.7-49.5 | M | 2 | 3 | 2-4 |
| 2 | 26.4-45.1 | F | 2 | 24 | 24 |
| 2 | 26.4-49.5 | M&F | 4 | 14 | 2-24 |
| 3 | 9.7-14.9 | M | 2 | 4 | 4 |
| 3 | 8.5-12.0 | F | 2 | 1.5 | 1-2 |
| 3 | 8.5-14.9 | M&F | 4 | 3 | 1-4 |
| 4 | 42.3-44.9 | M | 2 | 6 | 4-8 |
| 4 | 40.3-45.0 | F | 2 | 8 | 8 |
| 4 | 40.3-44.9 | M&F | 4 | 8 | 4-8 |

M . . . male
F . . . female

1. Pharmaceutical formulation comprising an LTB₄-antagonist of formula I 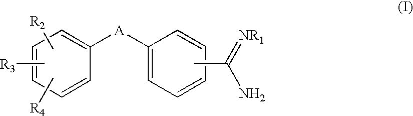
wherein
A denotes a group of formula II
—O—CₘH₂ₘ—O-(PHE)ₙ- (II)
wherein
m is an integer from 2 to 6,
n is 0 or 1,
PHE denotes a 1,4-phenylene group optionally substituted by one or two C₁-C₆-alkyl groups; or
A denotes a group of formula 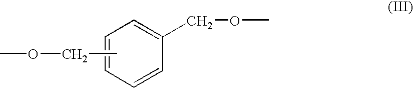
and wherein
R₁ denotes H, OH, CN, COR₁₀, COOR₁₀, or CHO;
R₂ denotes H, Br, Cl, F, CF₃, CHF₂, OH, HSO₃—O, C₁-C₆-alkyl, C₁-C₆-alkoxy, C₅-C₇-cycloalkyl, CONR₈R₉, aryl, O-aryl, CH₂-aryl, CR₅R₆-aryl, or C(CH₃)₂—R₇, wherein the aryl group denotes phenyl or naphthyl that may be mono- or polysubstituted by Cl, F, CF₃, C₁-C₄-alkyl, OH, HSO₃—O, or C₁-C₄-alkoxy;
R₃ denotes H, C₁-C₆-alkyl, C₁-C₆-alkoxy, OH, Cl, or F;
R₄ denotes H or C₁-C₆-alkyl;
R₅ denotes C₁-C₄-alkyl, CF₃, CH₂OH, COOH, or COO(C₁-C₄-alkyl);
R₆ denotes H, C₁-C₄-alkyl, or CF₃;
R₇ denotes CH₂OH, COOH, COO(C₁-C₄-alkyl), CONR₈R₉, or CH₂NR₈R₉;
R₈ denotes H, C₁-C₆-alkyl, phenyl, phenyl-(C₁-C₆-alkyl), COR₁₀, COOR₁₀, CHO, CONH₂, CONHR₁₀, SO₂—(C₁-C₆-alkyl), SO₂-phenyl, wherein the phenyl group may be mono- or di-substituted by Cl, F, CF₃, C₁-C₄-alkyl, OH, and/or C₁-C₄-alkoxy;
R₉ denotes H or C₁-C₆-alkyl; or
R₈ and R₉ taken together represent a C₄-C₆-alkylene group; and
R₁₀ denotes C₁-C₆-alkyl, C₅-C₇-cycloalkyl, aryl, heteroaryl, aralkyl, or heteroaryl-(C₁-C₆-alkyl), wherein the aryl group denotes phenyl or naphthyl, the heteroaryl group denotes pyrrole, pyrazole, imidazole, furanyl, thienyl, pyridine, or pyrimidine, and in each case may be mono- or polysubstituted by Cl, F, CF₃, C₁-C₄-alkyl, OH, HSO₃—O, or C₁-C₄-alkoxy,
or a pharmacologically acceptable acid addition salt, glycoside, O-sulphate, or glucuronide thereof,
wherein the LTB₄-antagonist is in the form of a solid solution or solid dispersion in a polymer matrix.

2. Pharmaceutical formulation according to claim 1, wherein the LTB₄ antagonist is [4-((3-((4-(1-(4-hydroxyphenyl)-1-methylethyl)phenoxy)methyl)benzyl)oxy)-benzenecarboximidamide-N-ethylcarboxylate] of formula IA:

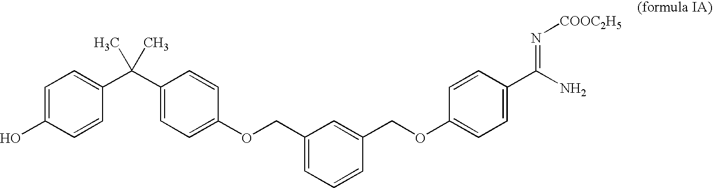

3. Pharmaceutical formulation according to claim 1, wherein the polymer matrix comprises one or more water-soluble polymers.

4. Pharmaceutical formulation according to claim 3, wherein the one or more water-soluble polymers are selected from the group consisting of: polyethyleneglycols, polypropyleneglycols, cellulose ethers, polyvinylpyrrolidones, polyvinyl acetates, copolymers, and mixtures thereof.

5. Pharmaceutical formulation according to claim 3, wherein the one or more water-soluble polymers are selected from the group consisting of: copolymers of polyethyleneglycols and polypropyleneglycols, methylcellulose, ethylcellulose, propylcellulose, carboxymethylcellulose, ethylhydroxyethylcellulose, hydroxypropylcellulose, N-vinylpyrrolidone homopolymers, mixed polymers of polyvinylpyrrolidone and polyvinyl acetate and polyethyleneglycols with various chain lengths.

6. Pharmaceutical formulation according to claim 3, wherein the polymer matrix is a poloxamer.

7. Pharmaceutical formulation according to claim 1, wherein the formulation contains about 0.5 to about 50 wt.-% of the LTB₄-antagonist based on the total weight of the pharmaceutical formulation.

8. Pharmaceutical formulation according to claim 1, wherein the formulation contains about 0.5 to about 25 wt.-% of the LTB₄-antagonist based on the total weight of the pharmaceutical formulation.

9. Pharmaceutical formulation according to claim 1, further comprising at least one excipient and/or carrier selected from the group consisting of: fillers, binders, disintegrants, breakdown agents, flow agents or flow regulators, lubricants, separators, pH correctors, antioxidants, and dyes.

10. Pharmaceutical formulation according to claim 9, wherein the proportion of excipients and/or carriers is within the range from about 50 to about 99.5 wt.-% based on the total weight of the pharmaceutical formulation.

11. Pharmaceutical formulation according to claim 9, wherein the proportion of excipients and/or carriers is within the range from about 90 to about 99 wt.-% based on the total weight of the pharmaceutical formulation.

12. Process for preparing a pharmaceutical formulation containing an LTB₄-antagonist of formula I, 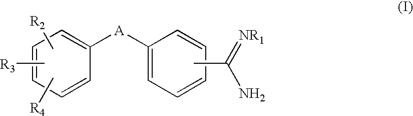
wherein
A denotes a group of formula II
—O—CₘH₂ₘ—O-(PHE)ₙ- (II)
wherein
m is an integer from 2 to 6,
n is 0 or 1,
PHE denotes a 1,4-phenylene group optionally substituted by one or two C₁-C₆-alkyl groups; or
A denotes a group of formula 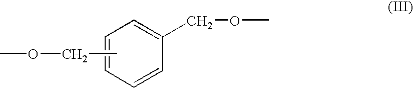
and wherein
R₁ denotes H, OH, CN, COR₁₀, COOR₁₀, or CHO;
R₂ denotes H, Br, Cl, F, CF₃, CHF₂, OH, HSO₃—O, C₁-C₆-alkyl, C₁-C₆-alkoxy, C₅-C₇-cycloalkyl, CONR₈R₉, aryl, O-aryl, CH₂-aryl, CR₅R₆-aryl, or C(CH₃)₂—R₇, wherein the aryl group denotes phenyl or naphthyl that may be mono- or polysubstituted by Cl, F, CF₃, C₁-C₄-alkyl, OH, HSO₃—O, or C₁-C₄-alkoxy;
R₃ denotes H, C₁-C₆-alkyl, C₁-C₆-alkoxy, OH, Cl, or F;
R₄ denotes H or C₁-C₆-alkyl;
R₅ denotes C₁-C₄-alkyl, CF₃, CH₂OH, COOH, or COO(C₁-C₄-alkyl);
R₆ denotes H, C₁-C₄-alkyl, or CF₃;
R₇ denotes CH₂OH, COOH, COO(C₁-C₄-alkyl), CONR₈R₉, or CH₂NR₈R₉;
R₈ denotes H, C₁-C₆-alkyl, phenyl, phenyl-(C₁-C₆-alkyl), COR₁₀, COOR₁₀, CHO, CONH₂, CONHR₁₀, SO₂—(C₁-C₆-alkyl), SO₂-phenyl, wherein the phenyl group may be mono- or di-substituted by Cl, F, CF₃, C₁-C₄-alkyl, OH, and/or C₁-C₄-alkoxy;
R₉ denotes H or C₁-C₆-alkyl; or
R₈ and R₉ taken together represent a C₄-C₆-alkylene group; and
R₁₀ denotes C₁-C₆-alkyl, C₅-C₇-cycloalkyl, aryl, heteroaryl, aralkyl, or heteroaryl-(C₁-C₆-alkyl), wherein the aryl group denotes phenyl or naphthyl, the heteroaryl group denotes pyrrole, pyrazole, imidazole, furanyl, thienyl, pyridine or pyrimidine and in each case may be mono- or polysubstituted by Cl, F, CF₃, C₁-C₄-alkyl, OH, HSO₃—O or C₁-C₄-alkoxy,
the method comprising the steps of:
(1) providing a melt comprising a melted polymer or a mixture of melted polymers;
(2) dissolving or dispersing an active substance comprising the LTB₄ antagonists of formula I in the melt to form a melted dispersion; and
(3) cooling the melted dispursion to form a solid solution or a solid dispersion.

13. Process according to claim 12, wherein the LTB₄ antagonist is [4-((3-((4-(1-(4-hydroxyphenyl)-1-methylethyl)phenoxy)methyl)benzyl)oxy)benzenecarboximid-amide-N-ethylcarboxylate] of formula IA:

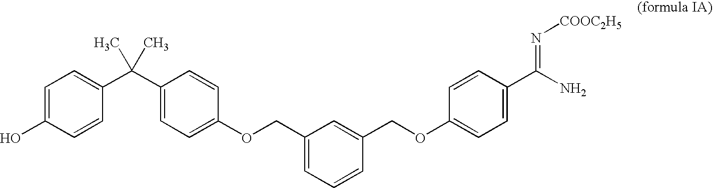

14. Process according to claim 12, wherein the polymer or mixture of polymers is selected from the group consisting of: polyethyleneglycols, polypropyleneglycols, cellulose ethers, polyvinylpyrrolidones, polyvinyl acetates, copolymers of polyethyleneglycols and polypropyleneglycols, methylcellulose, ethylcellulose, propylcellulose, carboxymethylcellulose, ethylhydroxyethylcellulose, hydroxypropylcellulose, N-vinylpyrrolidone homopolymers, mixed polymers of polyvinylpyrrolidone and polyvinyl acetate and polyethyleneglycols with various chain lengths.

15. Process according to claim 12, wherein the active substance in step (2) is used in crystalline, unground, ground, or jet-ground, or screened form.

16. Process according to claim 12, wherein the active substance used in step (2) has a mean particle size of about 1 μm to about 7 μm.

17. Process according to claim 12, wherein the active substance used in step (2) has a mean particle size of about 1.5 μm to about 3 μm.

18. Process according to claim 12, wherein step (3) further comprises pouring the melted dispersion into a suitably shaped mould before the melted dispersion cools.

19. Process according to claim 12, further comprising a step of (4) comminuting the solid solution or solid solution to obtain a suitable shape.

20. Process according to claim 12, wherein the the solid solution or the solid dispersion obtained from step (3) are packed into capsules.
